(19) United States
(12) Patent Application Publication
(10) Pub. No.: US 2025/0249051 A1
(43) Pub. Date: Aug. 7, 2025

(54) MICROBIOME COMPOSITION FOR IMPROVING MUSCLE STRENGTH USING HEAT-TREATED FERMENTED CULTURE COMPLEX OF LACTIPLANTIBACILLUS PLANTARUM KM2

(71) Applicant: KOOKMINBIO, CORP., Jeonju-si (KR)

(72) Inventor: Moon-Hee SUNG, Seoul (KR)

(73) Assignee: KOOKMINBIO, CORP., Jeonju-si (KR)

(21) Appl. No.: 19/001,393

(22) Filed: Dec. 24, 2024

(30) Foreign Application Priority Data
Feb. 6, 2024 (KR) 10-2024-0018228
Nov. 12, 2024 (KR) 10-2024-0160137

(51) Int. Cl. A61K 35/747; A61K 9/00; A61P 21/00; C12R 1/25

(52) U.S. Cl. CPC A61K 35/747; A61K 9/0053; A61P 21/00; C12R 2001/25

(57) ABSTRACT

The present disclosure relates to a microbiome composition for improving muscle strength using a heat-treated fermented culture complex of KM2 Lactiplantibacillus plantarum, wherein the composition according to the present disclosure increased synthesis of myoproteins and showed an effect of inhibiting degradation of myoproteins and animal tests showed an effect of improving muscle strength and increasing leg muscle mass through the increase in grip strength, travel distance, and speed, as well as results in which hamstring muscle strength is improved and muscle mass increases even in a clinical trial. In addition, it was found that the beneficial bacteria increased through an increase in the diversity of intestinal microflora that changed with muscle reduction and improvement in the microflora composition, while the harmful bacteria decreased.

### CROSS-REFERENCE TO RELATED APPLICATION

This application claims the benefit of Korean Patent Application No. 10-2024-0018228 filed on Feb. 6, 2024 and Korean Patent Application No. 10-2024-0160137 filed on Nov. 12, 2024, in the Korean Intellectual Property Office, the entire disclosure of which is incorporated herein by reference for all purposes.

### BACKGROUND

### 1. Field of the Invention

The present disclosure relates to a microbiome composition for improving muscle strength using a heat-treated fermented culture complex of KM2 Lactiplantibacillus plantarum.

### 2. Description of the Related Art

Muscle is the most abundant tissue in the human body, and it is essential to secure adequate muscle mass to maintain the functional ability of human body and prevent metabolic diseases. Muscle size is controlled by intracellular signaling processes that induce anabolism or catabolismtaking place in the muscle. When more signaling responses that induce synthesis of myoproteins occur over the breakdown, synthesis of myoproteins increases, resulting in muscle hypertrophy or an increase in the number of muscle fibers that brings an increase in the size of muscles.

Muscles also promote the influx of calcium to increase bone density. However, as the body becomes old, redistribution of body fat and body protein takes place due to changes in its composition, and at the age of about 50, the rate of protein synthesis in muscle cells slows down in comparison to the breakdown rate to cause rapid degeneration of muscles that might lead to muscopenia. Sarcopenia, a type of muscopenia, is a condition with reduction of about 13 to 24 percent of their usual body mass, resulting in a decrease in protein content, fiber diameter, production of muscle strength, and resistance to fatigue. Sarcopenia occurs by a variety of causes, including sepsis, cancer, kidney failure, excess glucocorticoids, nerve removal, muscle underuse, and the aging process.

The gradual decrease in the quantity and quality of skeletal muscle that occurs with aging and weight loss including fat and body fat components following the inadequate dietary energy intake may be the causes, with high correlation with age due to senescence. Sarcopenia arises from an imbalance between synthesis and breakdown of proteins. Sarcopenia also degrades life satisfaction and makes it vulnerable to injuries even in common daily life. In addition, intensive exercise causes muscle fatigue and damage and downgrades performance. Muscle injuries include bruises, lacerations, ischemia, strains, and severe damage to skeletal muscles. Such damage may cause a lot of pain. If there is severe damage to the skeletal muscle, measures that may reduce muscle damage or facilitate the recovery of muscle tissues may boost the recovery and muscle strength post-exercise. It may also help muscles recover after an illness.

With the recent increased interest in weight control, rapid weight loss may cause sarcopenia, regardless of age, and intense exercise can damage muscles. Therefore, research and efforts are being made on treatment of muscle loss caused by common muscopenia as well as muscle increases, and research is being conducted on the treatment of muscle diseases and muscle strengthening as well.

### SUMMARY

### Problem to be Solved by the Invention

Accordingly, an object of the present disclosure is to provide a composition for preventing, ameliorating, or treating a muscle disease by improving muscle strength or increasing muscle mass and a composition for improving muscle strength or increasing muscle mass, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

### Means for Solving the Problem

The present disclosure provides a pharmaceutical composition for preventing or treating a muscle disease, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

In addition, the present disclosure provides a health functional food composition for preventing or ameliorating a muscle disease, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

In addition, the present disclosure provides a health functional food composition for improving muscle strength or increasing muscle mass, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

### Effects of the Invention

The present disclosure relates to a microbiome composition for improving muscle strength using a heat-treated fermented culture complex of Lactibacillus plantarum KM2, wherein, as a result of clinical trials, it was determined after 12 weeks of ingestion of the heat-treated fermented culture complex of Lactibacillus plantarum KM2, through measurement of thigh muscle strength, that the thigh muscle flexor (hamstring muscle strength) significantly increased compared to a control group, resulting in improvement in the muscle strength. As a result of analyzing muscle mass, it was found that muscle mass increased in a test group compared to the control group after 12 weeks of ingestion and, in particular, as a result of analysis in which the age of subjects was designated in their 50s with rapid decline in the physical functions, it was determined that the muscle mass increased statistically significantly in the test group compared to the control group. Moreover, fecal flora analysis showed that 12 weeks of ingestion of the heat-treated fermented culture complex of Lactiplantibacillus plantarum KM2 increased the diversity of intestinal microflora in the test group compared to the control group, and significantly changed the composition of the intestinal microbiota as well. Furthermore, it was observed that Veillonella spp. which is a beneficial bacterium increased, while Shigella spp. which is a harmful bacterium decreased.

### BRIEF DESCRIPTION OF THE DRAWINGS

FIG. 1 shows results of analyzing cytotoxicity and myotube diameter in cell experiments.

FIG. 2 shows results of analyzing expression of myoprotein degradation genes and expression of myoprotein synthesizing proteins in cell experiments.

FIG. 3 shows results of analyzing the body composition and lean body mass using DEXA in animal experiments.

FIG. 4 shows results of analyzing the muscle weight and muscle function measurement in animal experiments.

FIG. 5 shows results of histological analysis in animal experiments.

FIG. 6 shows results of analyzing expression of signaling proteins involved in the muscle atrophy inhibition and myoprotein synthesis in animal experiments.

FIG. 7 shows results of analyzing intestinal microflora in animal experiments.

FIG. 8 shows a participation status and analysis groups of subjects for a clinical trial.

FIG. 9 shows changes in the left hamstring strength.

FIG. 10 shows average changes in the hamstring strength.

FIG. 11 shows changes in muscle mass (ASM/Height², g/m²).

FIG. 12 shows changes in muscle mass (ASM/Height², g/m²) of subjects in their 50s.

FIG. 13 shows changes in total scores (points) in SPPB.

FIG. 14 shows changes in gate speed (sec) in SPPB.

FIG. 15 shows changes in repeated chair stands (sec) in SPPB.

FIG. 16 shows changes in CRP (mg/L).

FIG. 17 shows a result of analyzing α-diversity (Shannon Index, PP set) of fecal flora in a clinical trial.

FIG. 18 shows results of analyzing β-diversity (Bray-Curtis dissimilarity, PP set) of fecal flora in a clinical trial.

FIG. 19 shows a result of analyzing changes in the abundance of test group-specific fecal flora (MaAsLin2) after 12 weeks of ingestion in a clinical trial.

### DETAILED DESCRIPTION

The present disclosure provides a pharmaceutical composition for preventing or treating a muscle disease, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

Preferably, in terms of intestinal microbiota composition changes, the pharmaceutical composition may increase Veillonella spp. which is a beneficial bacterium and decrease Shigella spp. which is a harmful bacterium, more preferably, the Veillonella spp. may be any one or more selected from the group consisting of Veillonella sp. S12025-13, Veillonella dispar, Veillonella nakazawae, and Veillonella atypica, and the Shigella spp. may be any one or more selected from the group consisting of Shigella sonnei, Shigella boydii, Shigella dysenteriae, and Shigella flexneri, but is not limited thereto.

Preferably, the muscle disease may be, but is not limited to, muscular atrophy, sarcopenia, atony, muscular dystrophy, myasthenia gravis, or amyotrophic lateral sclerosis.

Preferably, the pharmaceutical composition may strengthen or improve muscle strength of hamstring, which is responsible for flexion among femoral muscles, but is not limited thereto.

Preferably, the pharmaceutical composition may increase muscle mass of people in their 50s with a significant loss in muscle mass, but is not limited thereto.

The pharmaceutical composition of the present disclosure may be prepared using pharmaceutically compatible and physiologically acceptable adjuvants in addition to the active ingredient, and solubilizers such as excipients, disintegrating agents, sweeteners, binders, coating agents, leavening agents, lubricants, glydents, or flavoring agents may be used as the adjuvant. The pharmaceutical composition of the present disclosure may preferably be formulated as a pharmaceutical composition by including one or more pharmaceutically acceptable carriers in addition to the active ingredient for administration. Acceptable pharmaceutical carriers for compositions to be formulated as liquid solutions may be those that are sterilized and biosuitable and may be used by mixing saline, sterile water, Ringer's solution, buffered saline, albumin injection, dextrose solution, maltodextrin solution, glycerol, ethanol, and one or more of these components, and other conventional additives such as antioxidants, buffers, and bacteriostatic agents may be added as required. In addition, diluents, dispersants, surfactants, binders, and lubricants may be additionally added to formulate into injectable preparations such as aqueous solutions, suspensions, and emulsions, pills, capsules, granules, or tablets.

A medicinal preparation form of the pharmaceutical composition of the present disclosure may be granules, acids, coated tablets, tablets, capsules, suppositories, syrups, juices, suspensions, emulsions, drops or injectable liquids, and slow-release preparations of active compounds. The pharmaceutical composition of the present disclosure may be administered via a conventional mode through intravenous, intraarterial, intraperitoneal, intramuscular, intraarterial, intraperitoneal, intrasternal, transdermal, intranasal, inhalation, topical, rectal, oral, intraocular, or intradermal routes. The effective dose of the active ingredient in the pharmaceutical composition of the present disclosure refers to an amount required for the prevention or treatment of a disease. Thus, it may be adjusted according to various factors, including the type of disease, the severity of the disease, the type and content of active ingredients and other components contained in the composition, the type of formulation and the patient's age, weight, general health status, sex and diet, duration of administration, route of administration and secretion ratio of the composition, treatment periods, and concomitant drugs.

In addition, the present disclosure provides a health functional food composition for preventing or ameliorating a muscle disease, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

Preferably, in terms of intestinal microbiota composition changes, the pharmaceutical composition may increase Veillonella spp. which is a beneficial bacterium and decrease Shigella spp. which is a harmful bacterium, more preferably, the Veillonella spp. may be any one or more selected from the group consisting of Veillonella sp. S12025-13, Veillonella dispar, Veillonella nakazawae, and Veillonella atypica, and the Shigella spp. may be any one or more selected from the group consisting of Shigella sonnei, Shigella boydii, Shigella dysenteriae, and Shigella flexneri, but is not limited thereto.

Preferably, the muscle disease may be, but is not limited to, muscular atrophy, sarcopenia, atony, muscular dystrophy, myasthenia gravis, or amyotrophic lateral sclerosis.

In addition, the present disclosure provides a health functional food composition for improving muscle strength or increasing muscle mass, including a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient.

Preferably, the health functional food composition may strengthen or improve the muscle strength of hamstring, which is responsible for flexion among femoral muscles, but is not limited thereto.

Preferably, the health functional food composition may increase muscle mass of people in their 50s with a significant loss in muscle mass, but is not limited thereto.

The health functional food composition of the present disclosure may be provided in the form of powder, granules, tablets, capsules, syrup, or beverages, and the health functional food composition may be used in combination with other foods or food additives in addition to the active ingredient and used appropriately in accordance with conventional methods. The amount of the active ingredient mixed may be appropriately determined according to the purpose of use thereof, e.g. preventive, health or therapeutic treatment.

The active ingredient contained in the health functional food composition may be used in accordance with the effective dose of the pharmaceutical composition, but in the case of long-term intake for health and hygiene or for health control, it may be below the above range, and it is certain that the active ingredient may be used in an amount above the range since there is no problem in terms of safety.

There is no specific limitation on the types of health foods, the examples of which include meat, sausages, bread, chocolate, candy, snacks, confectionery, pizza, ramen, other noodles, chewing gum, dairy products including ice cream, various soups, beverages, tea, drinks, alcoholic beverages, and vitamin complexes.

Hereinafter, to help the understanding of the present disclosure, example embodiments will be described in detail. However, the following example embodiments are merely illustrative of the content of the present disclosure, and the scope of the present disclosure is not limited to the following example embodiments. The example embodiments of the present disclosure are provided to more completely explain the present disclosure to those of ordinary skill in the art.

### Experimental Example

The following experimental examples are intended to provide experimental examples commonly applied to each example according to the present disclosure.

### 1. Preparation of Heat-Treated Fermented Culture Complex of Lactiplantibacillus Plantarum KM2

Lactiplantibacillus plantarum KM2 was inoculated into MRS medium (2%, v/v) and cultured at 30° C. for 12 hours, followed by heat treatment at 90° C. for 1 hour. Cell-free supernatant of L. plantarum (CFS-L. plantarum) was collected by a centrifuge and filtered through a 0.2-μm membrane. CFS-L. plantarum was lyophilized with the addition of 7% (w/v) soybean flour, the centrifuged bacteria were dried, and 10⁹ cells/g of heat-treated microorganisms were added to the CFS powder to prepare a heat-treated fermented culture complex of Lactiplantibacillus plantarum KM2 (KLP-KM2).

### 2. Cell Experiments

### (1) C2C12 Cell Culture and Viability Measurement

Mouse C2C12 myoblasts (ATCC, Manassas, VA, USA) were passage-cultured at 37° C. in a 5% CO₂ incubator using DMEM containing 10% fetal bovine serum and 1% penicillin-streptomycin. C2C12 myoblasts were dispensed at 5×10³ cells/well in a 96-well plate, treated with KLP-KM2 (200, 400, 800 μg/mL), and cultured for 48 hours, followed by analysis on the cell viability using MTT assay.

### (2) C2C12 Cell Differentiation and Formation of Muscular Atrophy Model

To differentiate C2C12 myoblasts into myotube cells, the cells were dispensed at 8×10⁵ cells/well in a 6-well plate and replaced with DMEM medium containing 2% horse serum and 1% penicillin-streptomycin at 100% confluency for differentiation for 6 days. As shown in drawings, muscular atrophy samples were treated with dexamethasone (DEX: 100 μM), known as an inducer, to induce muscular atrophy in C2C12 cells that were differentiated into myotubes and treated with DEX, followed by analysis after culture for 24 or 48 hours.

### (3) Jenner-Giemsa Staining for Morphological Changes of C2C12 Cells

For morphological changes in muscle cells due to muscular atrophy, the diameter of the myotube was measured by Jenner-Giemsa staining. Differentiated C2C12 cells were stained using Jenner-Giemsa solution, and the fusion index (%) and myotube width were measured using DM IL LED microscopy (Leica Microsystems, Germany).

### (4) Analysis of Improvement in Muscular Atrophy Using C2C12 Myotubes

Since C2C12, a myoblast, differentiates into the myotube and increases the expression of MyoD and myogenin, the increase in the expression of myogenesis-related factors by sample treatment was analyzed using real-time quantitative PCR (q-RT PCR). As the expression of protein degradation factors FoxO3a, MuRF1, and Atrogin-1 was induced by the treatment of DEX, the inhibition of expression for protein degradation factors by sample addition was analyzed using q-RT PCR and Western blotting. Since muscular atrophy may be mediated by decreased activation of the mTOR/PI3K/Akt pathway, the increase in expression of protein synthesis factors mTOR, PI3K, and Akt by sample treatment was analyzed using real-time quantitative PCR (q-RT PCR).

### 3. Animal Tests

### (1) Preparation and Designing of Animal Test

7-week-old male C57BL/6J mice purchased from OrientBio were used as experimental animals. The environment of an animal breeding room was controlled at temperature of 21±1° C. with relative humidity of 40˜60% and light/dark cycles of 12 hours/day, and feed and drinking water were allowed to be consumed freely. This test was approved by the Institutional Animal Care and Use Committee in Kookmin University (KMU-2023-05) and carried out in accordance with the standard operating instructions. After the adaptation period, the experimental group was divided into a total of 5 groups with 8 mice each, the groups of which are a normal control group (normal; NOR), a negative control group (dexamethasone; DEX), a low-dose sample treated group (KLP-KM2 900 mg/kg; KBL), a high-dose sample treated group (KLP-KM2 1800 mg/kg; KBH), and a positive control group (50 mg/kg of oxymetholone; OXM). To create a muscular atrophy model, the DEX, KLP-KM2, and OXM groups were administered intraperitoneally with dexamethasone (5 mg/kg) between 10˜11 AM every day for 8 weeks. KLP-KM2 and OXM were administered orally for 8 weeks, while the NOR and DEX groups received normal saline orally during the same period. Feed ingestion amount and body weight were measured once every two weeks.

### (2) Analysis on Body Composition

Dual energy X-ray absorptiometry (DEXA; Medikors, Seongnam, Korea) was used at week 7 before the sacrifice of experimental animals to measure the body composition of mice, followed by analysis on the changes in muscle mass relative to the body weight.

### (3) Biochemical Analysis of Serum

After sacrificing the mice, blood was drawn from the heart and immediately centrifuged (3,000×g, 10 min, 4° C.) to separate the plasma. Creatine phosphokinase (CPK), lactate dehydrogenase (LDH), aspartate aminotransferase (AST), alanine aminotransferase (ALT), and blood urea nitrogen (BUN) were measured using a chemical analyzer (Fuji DRI-CHEM 3500i, Fuji Photo Film, Ltd., Tokyo, Japan).

### (4) Measurement of Grip Strength

The grip strength of mice was measured after muscle atrophy was induced using a grip strength meter (DBI, Co., Eumsung, Korea). A stainless steel T-bar was attached to a gauge to repeatedly measure the grip strength 5 times for each mouse by making the mice hold the T-bar with both front legs and pulling the tail at a constant speed, with the average value recorded except for the maximum and minimum values.

### (5) Evaluation of Motor Skills

Mouse motility was measured using a treadmill, and the recorded images were analyzed using the EthoVision video tracking system (Noldus Information Technology, Wageningen, Netherlands). For treadmill measurements, after 2 days of training, the mice were set to run for 10 minutes at a speed of 10 m/min with a 10° incline, and the speed increased by 2 m/min every 3 minutes until reaching a maximum speed of 30 m/min.

### (6) Histological Analysis

After fixing the gastrocnemius muscle in 4% formaldehyde, paraffin blocks were fabricated, and H&E staining was carried out. The cross-sectional area of muscle tissue was measured using KFBIO Slide Manager (KFBIO, Ningbo, China), and the cross-sectional area of muscle fibers (μm²) was measured at 20× magnification using ImageJ software (Version 1.8.0, National Institutes of Health, USA) to obtain 100 cross-sectional areas of representative images, which was then expressed in an average.

### (7) Analysis of Protein Expression Related to Muscle Breakdown and Synthesis

Muscle cells and tissues were homogenized using a bullet blender (Next Advance, Troy, NY, USA) in a radioimmunoprecipitation assay (RIPA) buffer containing 1% protease inhibitor and 1% phosphorylase inhibitor to be used in the experiment. The homogenized tissue was left at 4° C. for 50 minutes and centrifuged for 15 minutes at 4° C. and 15,000×g to obtain a supernatant. The same amount of protein was isolated from 12% SDS-PAGE and transferred to polyvinylidene fluoride membranes (Bio-Rad, Hercules, CA, USA). After blocking with 5% bovine serum albumin-containing Tris-buffered saline solution with Tween 20 (hereinafter referred to as TBST, 0.1%) which is a blocking buffer solution, reaction was carried out overnight at 4° C. with antibodies against AKT (Cat. 4691S), p-AKT (Cat. 4060S), mTOR (Cat. 2986S), p-mTOR (Cat. 5536S), p-Fox03a (Cat. 9466S), and β-actin (Cat. 4970S, Cell Signaling Technology). Horseradish peroxidase-labeled secondary antibody was subjected to a reaction at room temperature for 1 hour, followed by washing four times with TBST buffer. Protein bands were identified with enhanced chemiluminescence detection kits (BioRad, Hercules, CA, USA), and band strength was corrected with β-actin protein and quantified using Image Lab software 5.1 (BioRad).

### (8) Analysis of Gene Expression Related to Muscle Breakdown and Synthesis

RNA was extracted using NucleoZol reagent (Macherey-Nagel, Duren, Germany) from muscle cells, and RNA was extracted using TRIzol reagent (Invitrogen, Carlsbad, CA, USA) from the gastrocnemius muscle which is a muscle tissue. cDNA was synthesized using a High-Capacity RNA-to-cDNA Kit (Applied Biosystems), and qRT-PCR was performed via the StepOne Plus RT-PCR System (Applied Biosystems) using Taqman Gene Expression Master Mix (Applied Biosystems). Quantitative results were presented by the 2-ΔΔCT method (Livak & Schmittgen, 2001) compared to reference mRNA (GAPDH). The primers used in the analysis were: GAPDH (Mm99999915_g1), FoxO3a (Mm01185722_m1), atrogin-1 (Mm00499523_m1), and MuRF1 (Mm01185221_m1).

### (9) Analysis of Intestinal Microflora

To identify the effect of the sample on the intestinal microbiota of the animal model, the appendix was removed after animal sacrifice, DNA was extracted, and analysis was performed using Shotgun metagenome sequencing. The extracted DNA was subjected to adapter ligation using the Truseq Nano DNA prep kit for sequencing, and then 8 cycles of PCR were performed to create a library. The size of the library prepared was checked with the Tapestation4200 instrument, and the data was generated by sequencing the library prepared in a length of 450˜650 bp with the Nextseq2000 device. Using Trimmomatic v0.39 (AM Bolger, M Lohse, B Usadel, 2014) for raw reads, removal of low-quality reads and adapters was followed, and in order to eliminate PhiX sequences added during sequencing, trimmed reads were aligned to the PhiX reference genome (NC_001422.1) using BWA v0.7.17-r1188 (Li, Heng, 2013) and then removed with SAMtools v1.15.1 (Li, Heng, et al., 2009). Taxonomy classification was performed using Kraken2 v2.1.2 (Wood et al., 2019), and species abundance was estimated by Bracken v2.55 (Lu, Jennifer, et al., 2017). Alpha diversity was measured by the rtk v0.2.6.1 R package (Saary, Paul, et al., 2017) using the Shannon index, and beta diversity was analyzed by calculating weighted and unweighted UniFrac distances using the phyloseq v1.34.0 R package (McMurdie & Holmes, 2013).

### (10) Statistical Analysis

The data were expressed as mean±standard error, and the statistical significance of the experimental results was analyzed using SPSS statistics V. 28 (SPSS Inc., USA). First, the difference in the treatment area was determined by one-way ANOVA, and if a significant difference was detected (P<0.05), the significance was verified between the treatment groups by Duncan's multiple comparative test method.

### 4. Clinical Trial

### (1) Design

This clinical trial was designed as a 12-week, randomized, double-blind, placebo-controlled parallel trial. When a test subject, who voluntarily signed the consent form for the clinical trial, participated in the clinical trial, a demographic survey, medical and drug administration history, non-drug treatment history survey, lifestyle survey, physical examination, vital signs (blood pressure, pulse), physical measurement {height, weight, body mass index (BMI)}, clinical pathology test, pregnancy response test (only for women of childbearing age), electrocardiogram, skeletal muscle mass (Inbody), grip strength, and blood index tests were conducted, and if the selection/exclusion criteria were met, the test subject was enrolled by random assignment. Test subjects assigned to the test group or control group were asked to consume either the test food or the control food for a total of 12 weeks. The assigned ratio for each group was as test group:control group=1:1.

### (2) Selection of Candidates

Subjects who did not meet the exclusion criteria were selected for those aged 50˜85 years, with the grip strength less than 36 kg for men or less than 22 kg for women and the muscle strength decreased less than 100% of the standard range for skeletal muscle mass measured by Inbody.

### (3) Foods for Clinical Trial

The heat-treated fermented culture complex of Lactiplantibacillus plantarum KM2 (hereinafter referred to as KLP-KM2) and control food were manufactured by Kookmin Bio Co., Ltd. Subjects consumed the food in the form of small pills for 12 weeks, 9 g/day, twice a day, with 2 packets at one time along with sufficient water.

### (4) Random Assignment

This clinical trial was conducted as a parallel test by randomly assigning to either the test group or the control group, and the required number of subjects for the clinical trial was 80 people in total, with 40 people in each group, in consideration of the dropout rate (25%).

In the randomization visit (Week 0), all subjects of the clinical trial who are suitable to meet the selection/exclusion criteria were assigned to each group according to the allocation code of the block randomization method. For balanced random assignment among the ingestion groups, the ratio of the number of subjects in each group was the same as 1:1.

The random assignment table is a sequence of random numbers (of A and B) that were generated by the randomization program in the SAS® system and applied sequentially starting from subject number 1 for the clinical trial, which was designed and created in advance before the clinical trial through SAS®. The person requesting the clinical trial had a food label for the clinical trial attached according to the random assignment table when packaging the food for the trial, and then supplied it to the clinical trial institution before the start of the trial.

### (5) Measurement of Femoral Strength

Femoral muscle strength was measured with the strength of quadriceps and hamstring on visits 2, 3, and 4 at angular velocity of 60°/see using isokinetic assessment equipment (Con-Trex). Sitting on the chair of the muscle strength meter, while the trunk is fixed to the backrest and the knee joint axis of the leg to be measured is aligned with the axis of the dynamometer, the distal femur, ankle joint, and upper bone were firmly fixed using resistance pads. The motion range of the joints was determined as the maximum movable range.

The right and left sides were measured 5 times each, with 3 times repetition, and the maximum value of the measured quadriceps strength was recorded each time.

In the measurement of muscle strength in the lower limb through an isokinetic evaluation device (Con-Trex), the independent contraction of extensors (quadriceps muscles) and flexors (hamstrings) of the femoral muscles is mainly involved, and in this clinical trial study, the items for efficacy evaluation were divided into extensors (quadriceps muscle strength) and flexors (hamstring strength) of the femoral muscle strength, the results of which were collected.

### (6) Measurement of Muscle Mass (ASM/Height²)

The diagnostic criteria for sarcopenia were first proposed by Baumgartner in 1998 when he presented the results of the New Mexico Elder Health Survey (NMEHS). In this study, Baumgartner defined appendicular skeletal muscle mass (ASM) as the sum of muscle mass excluding bone mineral mass from lean body mass of the limbs among values measured by DEXA, and as the height increases, limb muscle mass increases, calculated by dividing by the square of height, such as body mass index (BMI). The lean body mass of the limbs was measured using DEXA at visits 2, 3, and 4, and the ASM value was obtained by subtracting bone mineral mass from the lean body mass of the limbs, which was then corrected by the square of the height.

### (7) Short Physical Performance Battery (SPPB)

The Short physical performance battery (SPPB), which was conducted on visits 2, 3 and 4, was first used by the National Institute of Aging (NIA) in the United States in a study to establish epidemiological studies in the elderly and has been used to easily assess the physical function of the elderly. It is a performance test that evaluates lower limb functions, consisting of 3 items including standing balance test, gait speed, and 5 times repeated chair stands, where each task is given a score of 0 points for inability to perform, 1 to 4 points depending on the performance, and a total of 12 points with 4 points for each task if succeeded in all tasks.
Standing balance: The balance test consists of sub-items of side-by side stance, semi-tandem stance, and tandem stance, where each posture is examined in order and evaluated after being demonstrated to the subject by an experienced examiner. If the side-by side stance was maintained for more than 10 seconds, 1 point was given, and if not, the next test, the gait speed test, was performed. If 1 point was given for the side-by side stance, the semi-tandem stance was tested, and if maintained for more than 10 seconds, 1 point was given, and the tandem stance was performed. If the tandem stance was maintained for more than 3 seconds, 1 point was given, 2 points were given if maintained for more than 10 seconds, while the perfect score for the balance test was 4.
Gait speed: The gate speed was evaluated by the time it took to walk a distance of 4 m, where 0 point was given if the person was instructed to walk at the usual pace and unable to do so, 1 point if exceeded 8.7 seconds, 2 points for 6.21˜8.7 seconds, 3 points for 4.82˜6.20 seconds, and 4 points for less than 4.82 seconds. The measurement was conducted 2 times in total and evaluated based on fast time, and the same method was used to test if the subject is able to walk using walking aids such as canes.
Repeated chair stands: Repeated chair stands were evaluated as the time it took to stand up and sit down on a chair 5 times with arms folded over chest, where 0 point was given for not completing the test within 60 seconds, 1 point for 16.7 seconds or more, 2 points for 13.7˜16.6 seconds, 3 points for 11.2˜13.6 seconds, and 4 points for 11.1 seconds or less. If the subject uses his or her hands or arms to stand up or if there are concerns about the subject's safety, such as a fall during the examination, the examiner may stop testing, and the subject will be given a score of 0. The aim is to assess the lower body strength required to perform many tasks, such as climbing stairs, walking, standing up from a chair or bath, and getting out of a car. If this exercise capacity increases, the likelihood and frequency of falls decrease.

### (8) CRP

C-Reactive Protein (CRP) is increased during a loss of skeletal muscle mass due to sarcopenia, and it has been reported that high levels of CRP are directly related to muscle functional decline.

Blood was collected and examined at visits 1 and 4.

### (9) Analysis of Intestinal Microflora

To determine the effect of the sample on the intestinal microflora, feces were collected after the end of sample administration followed by DNA extraction, and analysis was performed using Shotgun metagenome sequencing. The extracted DNA was subjected to adapter ligation using the Truseq Nano DNA prep kit for sequencing, and then 8 cycles of PCR were performed to create a library. The size of the library prepared was checked with the Tapestation4200 instrument, and the data was generated by sequencing the library prepared in a length of 450˜650 bp with the Nextseq2000 device. Using Trimmomatic v0.39 (AM Bolger, M Lohse, B Usadel, 2014) for raw reads, removal of low-quality reads and adapters was followed, and in order to remove PhiX sequences added during sequencing, trimmed reads were aligned to the PhiX reference genome (NC_001422.1) using BWA v0.7.17-r1188 (Li, Heng, 2013) and then removed with SAMtools v1.15.1 (Li, Heng, et al., 2009). Taxonomy classification was performed using Kraken2 v2.1.2 (Wood et al., 2019), and species abundance was estimated by Bracken v2.55 (Lu, Jennifer, et al., 2017). Alpha diversity was measured by the rtk v0.2.6.1 R package (Saary, Paul, et al., 2017) using the Shannon index, and beta diversity was analyzed by calculating weighted and unweighted UniFrac distances using the phyloseq v1.34.0 R package (McMurdie & Holmes, 2013).

### (10) Adverse Events

Safety set analysis was conducted as the main analysis for adverse events, and 40 people for the test group and 40 people for the control group were included in the analysis as the subjects for the clinical trial who consumed food for the clinical trial at least once after being randomly assigned to the clinical trial. The type and incidence of adverse events, the severity of symptoms, and the association with the food for clinical trial were evaluated.

### (11) Statistical Analysis Method

Statistical analysis was performed using SAS® (Version 9.4, SAS Institute, Cary, North Carolina, USA).

Data on effectiveness, demographic and nutritional analysis data, and data on safety were set to a significant level of 0.05, followed by a two-tail test. The p-values of all analyses were presented to 4 decimal places, and the p-values of all analyses were considered significant if they were <0.05. For values with decimal values such as mean, standard deviation, and percentage, up to 2 decimal places were presented.

For the comparison among groups, the test group and the control group were subjected to the comparative analysis using two sample t-test when the normality was satisfied at p-value of 0.05 via the normality assay (Kolmogorov-Smirnov), and the comparison analysis was conducted using the Wilcoxon rank sum test when the normality was not satisfied in any of the groups. For the comparison among groups, comparative analysis was performed via a normality assay (Kolmogorov-Smirnov) using a paired t-test when normality was satisfied, and analysis was conducted using a Wilcoxon signed rank test when the normality was dissatisfied. ANCOVA was performed using the baseline of each variable as a covariate.

### <Example 1> Cell Experiment

### 1. Analysis of Cytotoxicity and Myotube Diameter

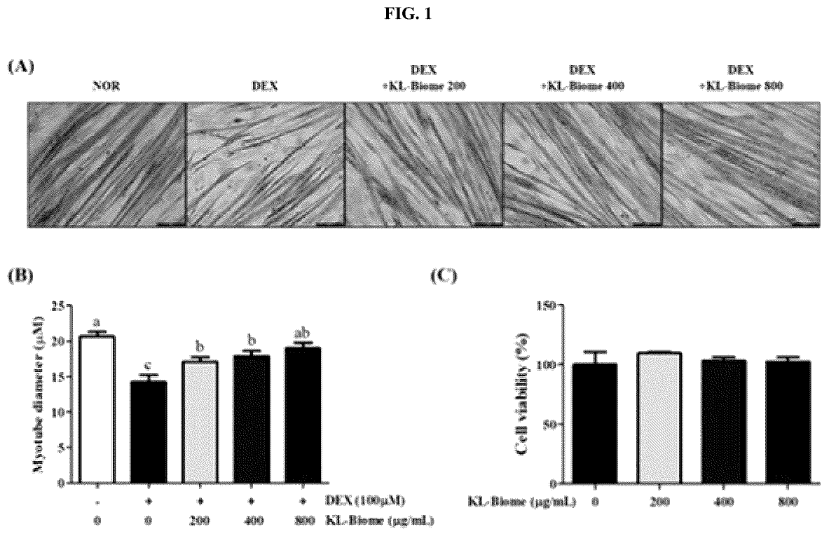

As a result of analyzing the cytotoxicity of KLP-KM2 samples in muscle cells, it was found that there was no significant change in the cell viability up to 800 μg/mL of samples. Subsequently, treatment of 200-800 μg/mL sample significantly increased the myotube diameter that was reduced by DEX, compared to the control group, with a 33% increase at the highest concentration of 800 μg/mL (FIG. 1).

### 2. Analysis on Expression of Myoprotein Degradation Genes and Expression of Myoprotein Synthesis Protein

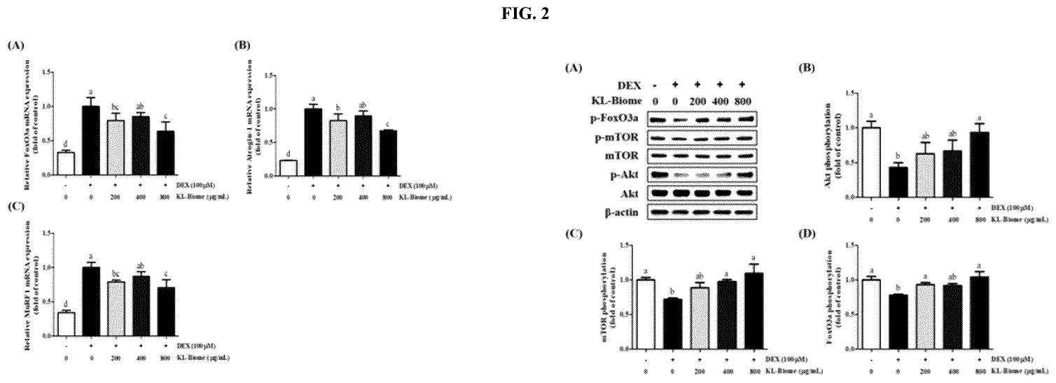

As a result of analyzing the effect of KLP-KM2 samples on the factors of myoprotein degradation and synthesis in muscle cells, the samples significantly reduced the gene expression of myoprotein degradation factors FoxO3a, Atrogin-1, and MuRF1 in a concentration-dependent manner, by 36%, 33%, and 30%, respectively, at the highest concentration of 800 μg/mL. The protein expression of Akt, mTOR, and Fox03a, which are myoprotein synthesizing factors, was also significantly increased in a concentration-dependent manner. In addition, from the above results, it was found that KLP-KM2 has the effect of improving muscle loss by inhibiting myoprotein degradation genes and increasing myoprotein synthesis at the cellular level (FIG. 2).

As a result of cell experiments, KLP-KM2 (KB) significantly increased the reduced myotube diameter in muscle cells induced with muscle atrophy by dexamethasone, promoted myoprotein synthesis, and inhibited myoprotein degradation.

### <Example 2> Animal Experiment

### 1. Analysis on the Body Composition and Lean Body Mass Using DEXA

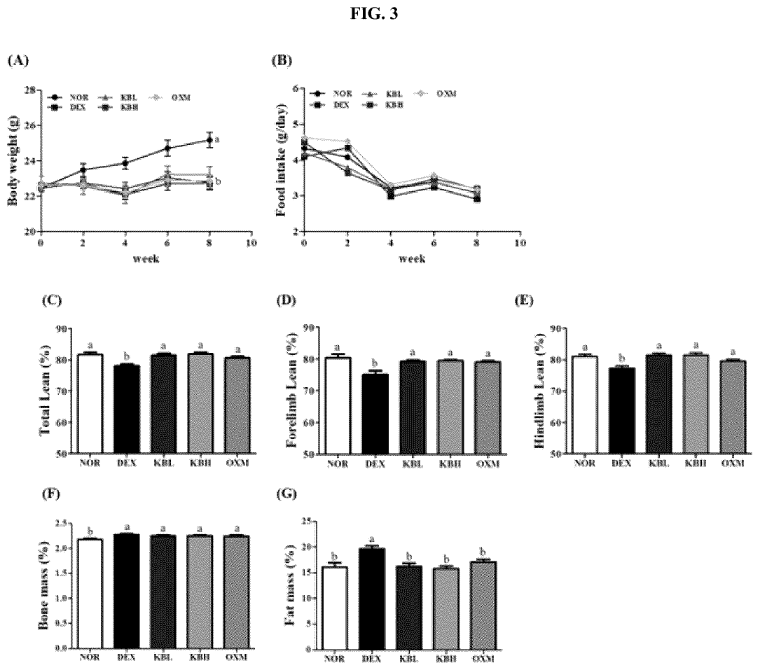

To assess the efficacy in inhibition of muscle loss, as a result of measuring the body weight and food ingestion by assessing the efficacy in the dexamethasone-induced animal, there was a significant reduction in body weight in all DEX treated groups compared to the NOR group, with no significant difference in food ingestion between groups. As a result of measuring the change in the body composition using DEXA, the DEX group showed a significant decrease in the muscle mass to the total body weight compared to the NOR group, while the KLP-KM2 group (KB900, KB1800) and OXM group showed a relatively significant increase compared to the DEX group. Hind and forelimb muscles were significantly reduced in all DEX treated groups, but there was no significant difference between DEX groups (FIG. 3).

### 2. Analysis of Muscle Weight and Muscle Function Measurements

As a result of measuring the weight of the mouse gastrocnemius muscle and quadriceps muscle immediately after animal sacrifice, the weight of the gastrocnemius muscle and quadriceps muscle increased significantly in the KLP-KM2 group in a concentration-dependent manner compared to the DEX group.

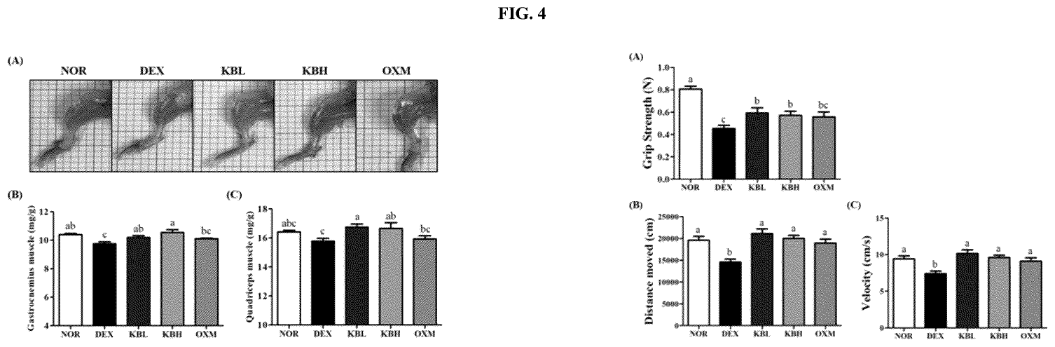

To evaluate whether the administration of the sample brings improvement in the muscle function decline in DEX-induced animal models, the muscle strength and running performance in the forelimbs of mice were evaluated using a grip strength meter and a treadmill. The KLP-KM2 group maintained significantly higher muscle strength compared to the DEX group. As a result of the treadmill exercise, the DEX group showed a significantly decrease in the running distance and speed compared to the NOR group during the same period, but the KLP-KM2 group had a significantly increase compared to the DEX group and maintained similar motor ability to the NOR group, deriving an improvement in muscle functions of KLP-KM2 (FIG. 4).

### 3. Histological Analysis

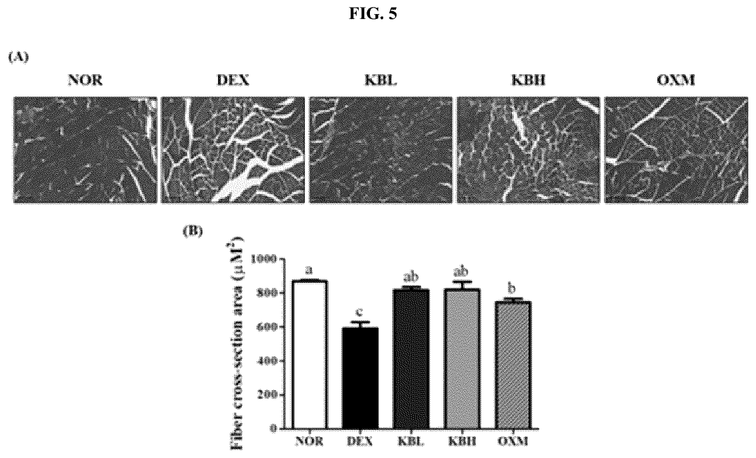

Since atrophy of muscle fibers is used as an important indicator of muscle damage, the gastrocnemius muscle was stained with H&E to assess the structural changes in the muscles by sample administration, and then the cross-sectional area of the muscle was observed. As a result, the DEX group showed a decrease in the size of muscle fibers and an increase in the gap between muscle fibers compared to the NOR group, and the KLP-KM2 group mitigated muscle tissue damage compared to the DEX group. In order to quantify morphological changes in muscle fibers, as a result of measuring the CSA of muscle fibers, the DEX group showed a significant decrease in CSA compared to the NOR group, and the KLP-KM2 group showed a significant recovery of CSA. As a result, the cross-sectional area of the muscle was maintained at a level similar to that of muscular dystrophy by the ingestion of KLP-KM2 (FIG. 5).

### 4. Biochemical Analysis of Serum

ALT and AST are biomarkers that are frequently used to assess hepatotoxicity, and DEX administration has resulted in an increase in ALT and AST. As a result of analysis on serum levels of ALT, AST, and BUN to assess the in vivo toxicity of KLP-KM2, a significant increase was observed in AST in the DEX group compared to the NOR group, and there was also an increase in ALT levels. However, there was no significant difference in the KLP-KM2 group compared to the DEX group. BUN is a commonly used biomarker to assess nephrotoxicity, and measurements showed no significant increase in the BUN levels in the KLP-KM2 group compared to the NOR group. These results suggest that KLP-KM2 administration does not cause hepatotoxicity or nephrotoxicity in mice, with a partial protective effect against DEX-induced liver and kidney damage (Table 1).

TABLE 1

| Group | CPK (U/L) | LDH (U/L) | ALT (U/L) | AST (U/L) | BUN (mg/dL) |
|---|---|---|---|---|---|
| NOR | 187.00 ± 62.34 ^{b} | 288.50 ± 73.19 ^{a} | 25.25 ± 6.88 ^{c} | 38.88 ± 9.31 ^{b} | 24.60 ± 2.76 ^{a} |
| DEX | 340.88 ± 154.51 ^{a} | 295.63 ± 65.91 ^{a} | 31.13 ± 2.30 ^{bc} | 59.25 ± 10.07 ^{a} | 24.15 ± 1.57 ^{a} |
| KBL | 147.50 ± 101.33 ^{b} | 99.25 ± 15.74 ^{c} | 29.00 ± 5.35 ^{bc} | 52.88 ± 10.22 ^{a} | 21.39 ± 1.13 ^{b} |
| KBH | 199.63 ± 56.45 ^{b} | 137.00 ± 18.59 ^{c} | 32.00 ± 3.42 ^{b} | 62.75 ± 12.28 ^{a} | 22.90 ± 1.09 ^{ab} |
| OXM | 99.13 ± 35.13 ^{b} | 211.38 ± 46.99 ^{b} | 43.75 ± 7.96 ^{a} | 58.88 ± 11.73 ^{a} | 24.74 ± 1.47 ^{a} |

### 5. Expression of Signaling Proteins Involved in Muscular Dystrophy Inhibition and Myoprotein Synthesis

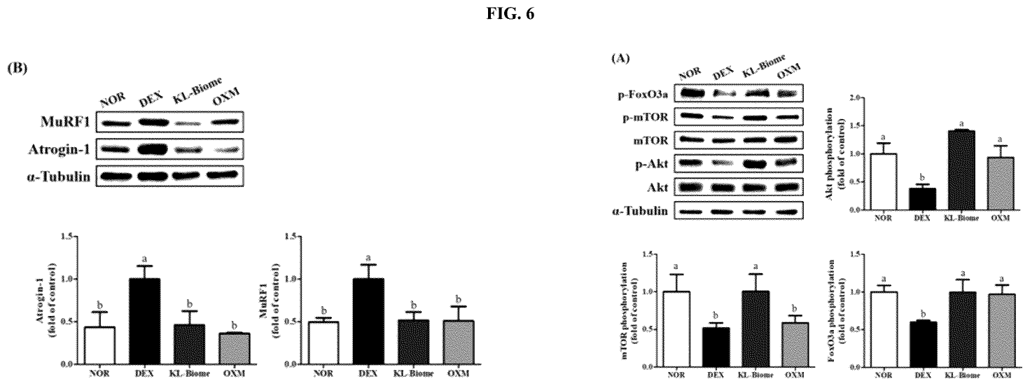

As a result of analyzing the effect of KLP-KM2 samples on factors of myoprotein degradation and synthesis in gastrocnemius muscle tissues, the expression of protein degradation factors Atrogin-1 and MuRF1 decreased significantly in the KLP-KM2 ingested group compared to the DEX group, and the expression of protein synthesis-related factors Akt, mTOR, and FoxO3a increased significantly compared to the DEX group. In addition, KLP-KM2 ingestion showed effective improvement in the control of DEX-induced muscle loss (FIG. 6).

### 6. Analysis of Intestinal Microflora

An F/B ratio of firmicutes and Bacteroidetes has been suggested as an important indicator of the health of the intestinal microflora, and the F/B ratio decreased significantly in the DEX group similar to the results reported that the ratio of F/B was remarkably reduced in chronic liver disease patients with low muscle mass, while the ratio was significantly increased by KLP-KM2 ingestion,

Subdoligranulum regulates abnormal inflammatory and immune conditions and may have a positive effect on muscle mass due to butyrate and its metabolites by butyrate-producing microorganisms. At the genus level, the relative abundance of Subdoligranulum was significantly reduced in the DEX group and significantly increased by KLP-KM2 administration.

The genus Alistipes are microorganisms present in the gastrointestinal tract of healthy humans, which are considered to be an important indicator of imbalance in the intestinal microbiota when depleted, and at the genus level, Alistipes was significantly reduced in the DEX group and significantly increased by KLP-KM2 administration.

It has been reported that the abundance of Akkermansia was increased in patients suffering from sarcopenia among senile chronic patients, and the proportion of Akkermansia at the genus level was significantly reduced in the KLP-KM2 and OXM groups compared to the DEX group.

Porphyromonas gingivalis is involved in elevation of blood sugar in people with type 2 diabetes, which is known to impair insulin sensitivity to cause sarcopenia. Ingestion of KLP-KM2 showed an effect of reducing Porphyromonas gingivalis at the species level, which was similar to that of the NOR group.

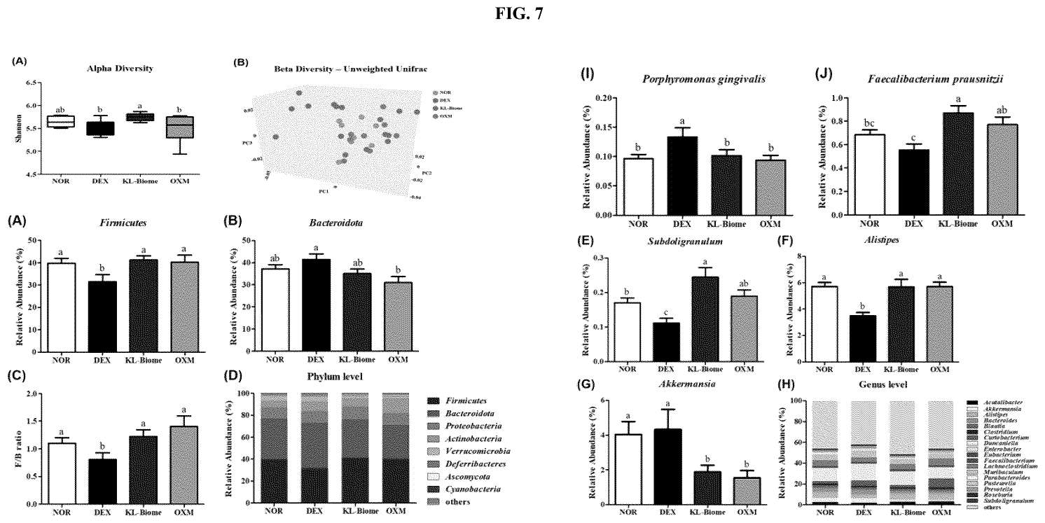

Faecalibacterium prausnitzii, which is known as a microorganism that produces SCFA, is known to increase gastrocnemius muscle mass and increase the expression of mitochondrial respiratory chain protein ATP5A as a result of administration to rats fed with a high-fat diet, and the relative abundance of Faecalibacterium prausnitzii at the species level was highest by KLP-KM2 ingestion (FIG. 7).

As a result of animal experiments, KLP-KM2 (KB) showed a significant increase in muscle weight (gastrocnemius, quadriceps) and lean body mass by KLP-KM2 administration in an animal model induced with muscular atrophy by dexamethasone, and significantly high muscle strength as well as the same level of motor ability as the normal group were maintained, showing improvement in muscle functions. As a result of histological analysis, in terms of the cross-sectional area of muscle, the same level of muscular dystrophy as in the normal group was shown. The intestinal microbiome showed increased microbial diversity due to KLP-KM2 administration, increased abundance in microorganisms that increase SCFA production, and decreased muscular dystrophy-related microorganisms.

In conclusion, in a C57BL/6J rodent model induced muscular atrophy by DEX treatment, administration of KLP-KM2 sample positively modulated muscular atrophy-related biomarkers by increasing lean body mass, muscle weight, grip strength, running speed, cross-sectional area of muscle tissues, and myoprotein synthesis factors and decreasing myoprotein degradation-related factors. Overall, the ingestion of KLP-KM2 is determined to be a postbiotic preparation that is effective in improving muscle loss by modulating intestinal microbiota.

### <Example 3> Clinical Trial

### 1. Selection of Test Subjects to be Included in Analysis

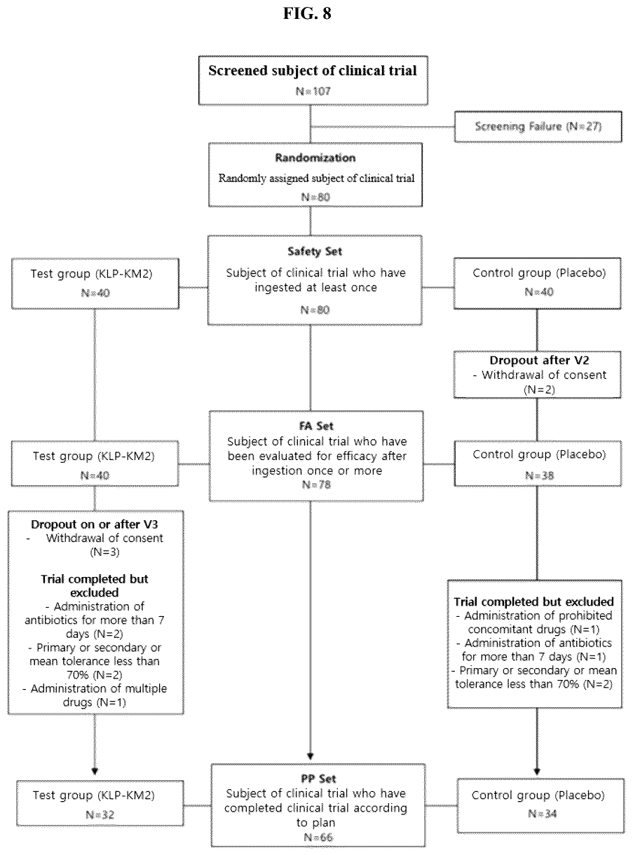

In FIG. 8, summarized are the participation status of subjects for the clinical trial and the organization of analysis groups from the beginning to the end of the clinical trial.

### 2. Demographic Information and Other Pre-Ingestion Characteristics

The aim is to determine different factors by comparing all the characteristics of each ingestion group before ingestion, including the demographic information of the subjects participated in the clinical trial.

As a result of examining the demographic information and characteristics of the subjects of the clinical trial before ingestion, in terms of gender, 4 males (12.50%) and 28 females (87.50%) were included in the test group, and 4 males (11.76%) and 30 females (88.24%) were included in the control group, with no statistically significant difference shown between ingestion groups.

In terms of age, the mean age was 58.97±6.48 years in the test group and 60.00±6.42 years in the control group, with no statistically significant difference between the ingestion groups.

In addition, there were no statistically significant differences in height, body weight, and BMI between the ingestion groups, such that comparisons between groups could be assumed (Table 2).

TABLE 2

| | Test group | Control group | |
|---|---|---|---|
| Variables | (n = 32) | (n = 34) | p-value |
| Gender | | | |
| Male | 4 | 4 | 1.0000(F)¹ |
| Female | 28 | 30 | |
| Age (yr) | 58.97 ± 6.48 | 60.00 ± 6.42 | 0.5500(W)² |
| Height (cm) | 157.44 ± 6.31 | 158.07 ± 5.46 | 0.3723(W)² |
| Body weight (kg) | 57.49 ± 7.57 | 56.56 ± 6.87 | 0.6054(T)² |
| BMI(kg/m2) | 23.16 ± 2.36 | 22.60 ± 2.06 | 0.3081(T)² |

¹Compared between groups; p-value for Fisher's exact test(F)
²Compared between groups; p-value for Two sample t-test(T) or Wilcoxon rank sum test(W)

### 3. Assessment of Effectiveness in Femoral Strength

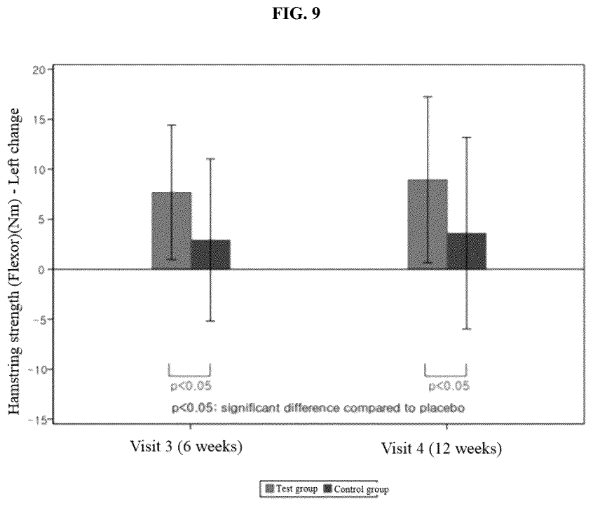

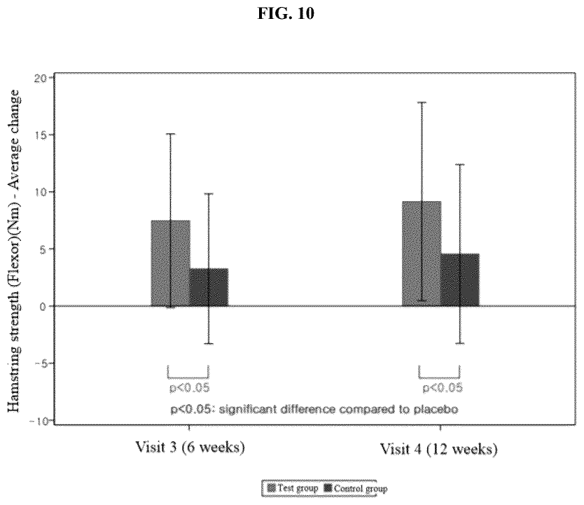

In this clinical trial study, it was found that the mean strength of the hamstring located at the posterior femur among the femoral muscles increased significantly after 12 weeks of ingestion, and there was a statistically significant increase in the left hamstring strength in the test group compared to the control group. After ingestion of the heat-treated fermented culture complex of Lactiplantibacillus plantarum KM2, there was an increase in the right hamstring strength, but no significant change was observed in the muscle strength in the right leg that is mainly used, suggesting that the increase in muscle strength in the left leg with relatively low usage is determined to be statistically significant (FIG. 9 and FIG. 10).

### 4. Muscle Mass, Simple Physical Performance Assessment, CRP, and Efficacy Assessment of Intestinal Microbiota

In the analysis on changes in muscle mass, after 6 weeks of ingestion, the test group showed an increase by 57.66±130.31 g/m² (p=0.0178), and the control group by 0.78±138.26 g/m² (p=0.9738). After 12 weeks of ingestion, the test group showed an increase by 17.01±137.13 g/m² (p=0.4882) while the control group showed a decrease by 30.17±118.23 g/m² (p=0.1463), indicating a positive tendency.

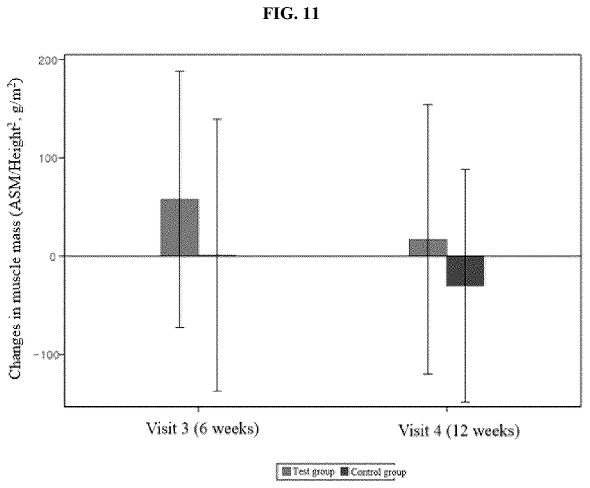

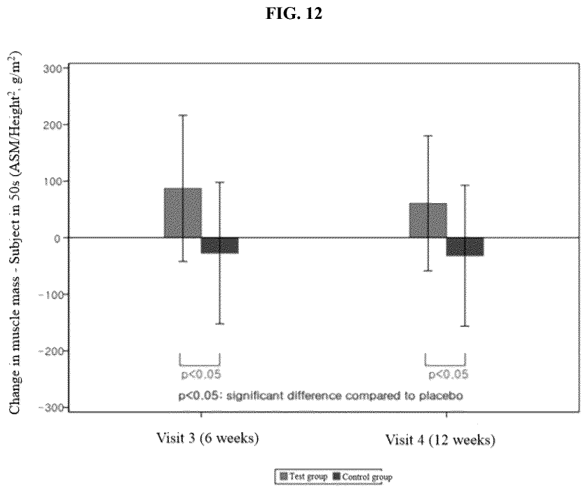

As a result of further analysis on the muscle mass by specifying the age of the subjects in their 50s with rapid decline in their physical functions, a statistically significant difference was observed with an increase by 60.63±119.44 g/m² in the test group and a decrease by 31.89±124.55 g/m² in the control group after 12 weeks of ingestion. This means that in the 50s, there was a significant increase in limb muscle mass in the test group compared to the control group (FIG. 11 and FIG. 12).

In the case of SPPB, the analysis on the change in the total SPPB score showed an increase by 0.03±0.31 points (p=1.0000) in the test group and a decrease by 0.03±0.17 points in the control group (p=1.0000) after 6 weeks of ingestion, but there was no statistically significant difference between the ingestion groups. After 12 weeks of ingestion, the test group showed an increase by 0.06±0.25 points (p=0.5000), and there was no change in the control group.

In the analysis on changes in the gate speed in SPPB, 6 weeks after ingestion, the test group showed an increase by 0.03±0.18 sec (p=1.0000) and the control group showed a decrease by 0.03±0.17 sec (p=1.0000), but there was no statistically significant difference between the ingestion groups. After 12 weeks of ingestion, the test group had an increase by 0.03±0.18 sec (p=1.0000), while there was no change in the control group.

In the analysis on changes in repeated chair stands in SPPB, 6 weeks after ingestion, the test group had an increase by 0.03±0.18 sec (p=1.0000), and there was no change in the control group. After 12 weeks of ingestion, the test group had an increase by 0.03±0.18 sec (p=1.0000), and there was no change in the control group.

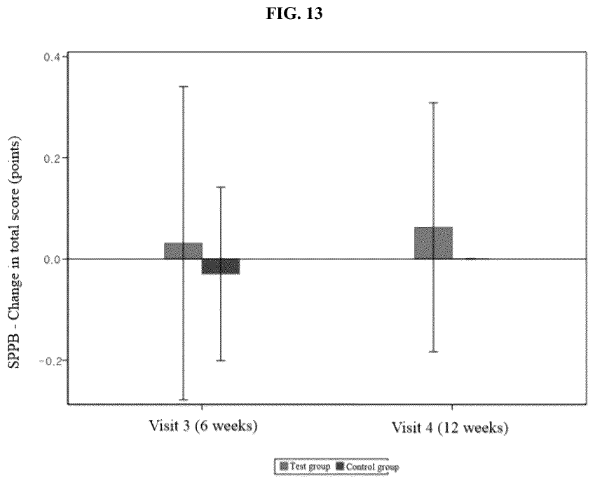

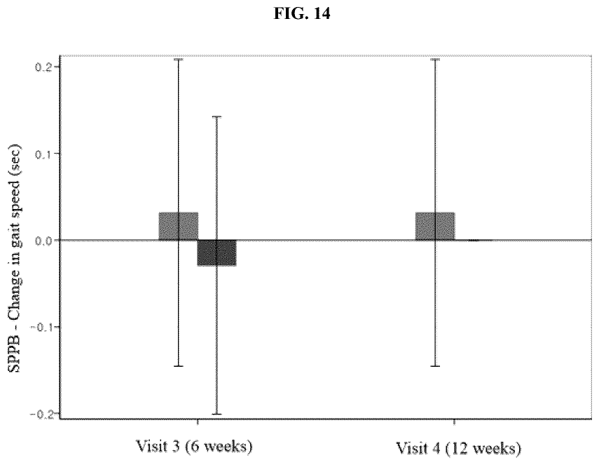

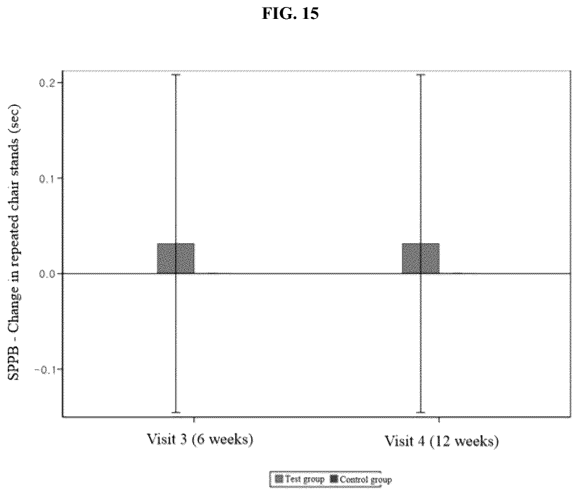

Since most subjects were rated with almost perfect scores at baseline, there was difficulty in determining the statistically significant change between the test and control groups after 6 and 12 weeks of ingestion, but there was no change in the control group and an increase in the test group after 12 weeks of ingestion. This is expected to be determined by further studies mediating SPPB (FIG. 13 to FIG. 15).

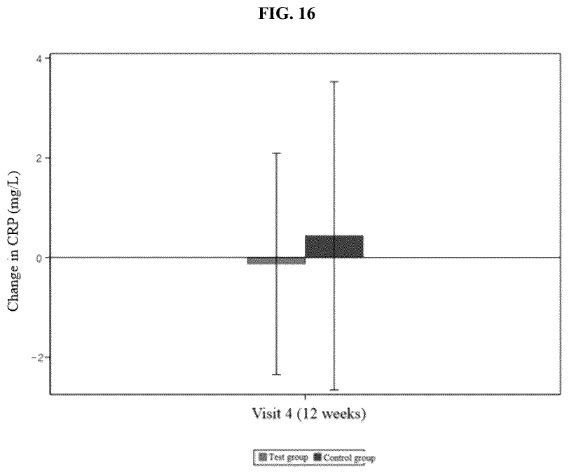

In the analysis on changes in CRP, measurement was carried out at baseline at 12 weeks, and subjects with CRP results of ‘<0.2’ measured at 12 weeks were excluded from the analysis. After 12 weeks of ingestion, the test group had a decrease by 0.13±2.22 mg/L (p=0.0554) and the control group had an increase by 0.43±3.09 mg/L (p=0.8516), showing tendency to increase significantly in the test group compared to the control group (FIG. 16).

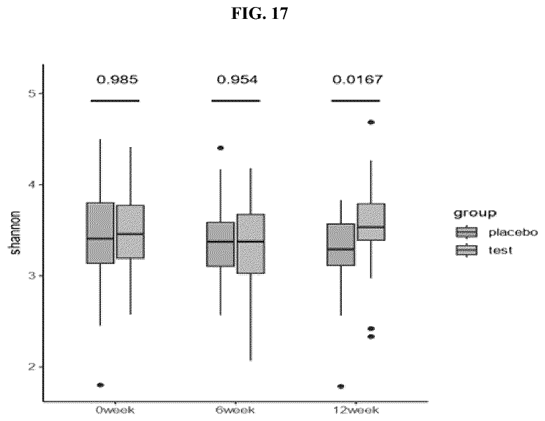

In the result of analyzing the diversity in the composition of microbial flora in feces by α-diversity (Shannon Index), the test group and the control group showed the same diversity at baseline, but after 12 weeks of ingestion, high diversity was observed at a statistically significant level in the test group compared to the control group (FIG. 17).

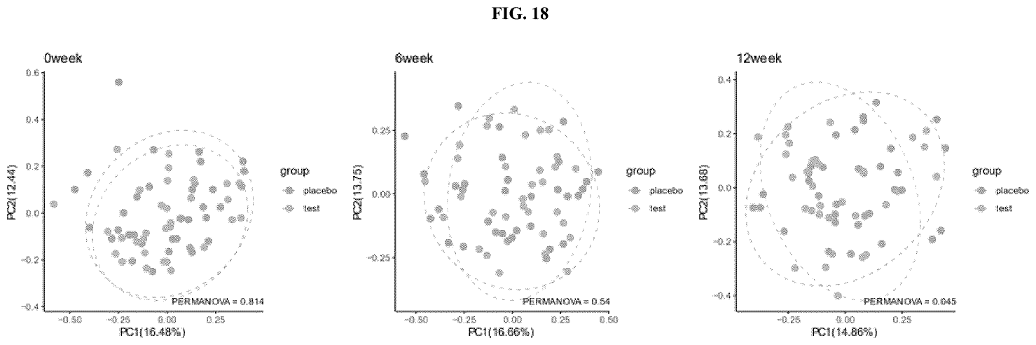

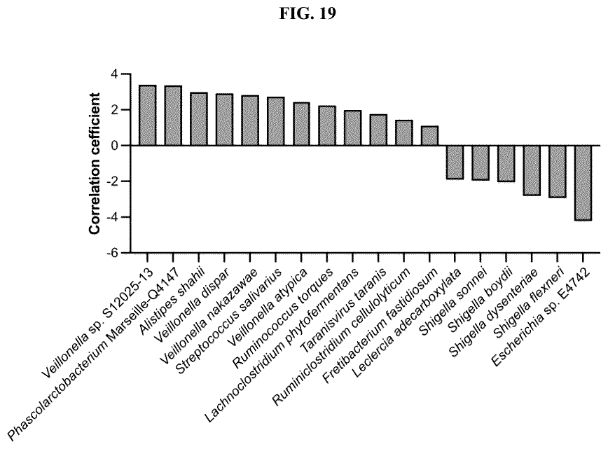

In the result of analyzing the structural differences in microbial flora in feces by β-diversity (Bray-Curtis dissimilarity), the test group and control group showed almost the same structure at baseline, but after 12 weeks of ingestion, the structure of the intestinal microflora of the control group and the test group was statistically significantly distinctive (FIG. 18). In the result of further analysis of fecal flora 12 weeks after ingestion using a microbiome multivariable association with linear models (MaAsLin2) based on a general linear model, there was statistically significant detection of strains with positive and negative correlations in the test group 12 weeks after ingestion (p<0.05). Among strains with positive correlations, a significant increase was observed in the genus Veillonella, and among the strains of the genus Veillonella, Veillonella atypica has been reported to be a bacterium that utilizes lactic acid. In addition, a significant reduction was found in the genus Shigella among strains with negative correlation, and Shigella has been reported to cause diarrhea and intestinal mucosal damage, which may hinder nutrient absorption and disrupt the supply of essential nutrients for muscle recovery and growth. In addition, the systemic inflammatory response caused by infection may accelerate the breakdown of myoproteins and exacerbate muscle damage (FIG. 19).

In conclusion, in this clinical trial study, a statistically significant increase was observed in the mean strength of hamstring located at the posterior femur among the femoral muscles after 12 weeks of ingestion, as well as a statistically significant increase in the left hamstring strength in the test group compared to the control group. Through the fecal flora analysis, it was determined that 12 weeks of ingestion of the heat-treated fermented culture complex of Lactiplantibacillus plantarum KM2 increased the diversity of intestinal microflora in the test group compared to the control group, and the composition of the intestinal microbiota of the test group and control group also changed significantly. In addition, the beneficial bacterium Veillonella spp. increased, among which Veillonella atypica is reported to be a bacterium that utilizes lactic acid. A decrease in harmful bacterium Shigella spp. was observed. Shigella has been reported to cause diarrhea and intestinal mucosal damage, which may impair nutrient absorption and disrupt the supply of essential nutrients for muscle repair and growth. In addition, the systemic inflammatory response caused by infection accelerates the breakdown of myoproteins and exacerbates muscle damage. In addition, as a result of further analysis on the muscle mass by specifying the age of the subjects of the clinical trial in their 50s with rapid decline in the physical function, it was found that it increased in the test group after 12 weeks of ingestion and decreased in the control group, showing a statistically significant difference. This indicates that there was a significant increase in the muscle mass in limb in the test group in the 50s compared to the control group, identifying the effect of the heat-treated fermented culture complex of Lactiplantibacillus plantarum KM2 on the improvement in the muscle strength and increase in muscle mass.

### 5. Adverse Events

In the test group, there were 22 adverse events in a total of 13 subjects (32.50%) of the clinical trial and 15 adverse events in a total of 13 subjects (32.50%) of the clinical trial in the control group, with no statistically significant difference between the ingestion groups.

In the survey for the severity of the symptom in adverse events that occurred during the clinical trial, the test group had 22 mild cases, and the control group had 15 mild cases.

In terms of relevance to food for clinical trial, the test group had 4 cases of ‘possibly related’ and 18 cases of ‘definitely not related’, while the control group had 2 cases of ‘probably not related’ and 13 cases of ‘definitely not related’, which was determined by examiner.

In the test group, 1 serious adverse event (urinary tract infection) occurred in 1 subject (2.50%) of the clinical trial and there were no dropouts due to adverse events (Table 3)

TABLE 3

| | | Test group | Control group |
|---|---|---|---|
| | | N = 40 | N = 40 |
| Adverse events n (%) | Yes | 13 (32.50) | 13 (32.50) |
| | No | 27 (67.50) | 27 (67.50) |
| | p-value [1] | | 1.0000(C) |
| Degree of adverse events n, | Mild | 13, 22 | 13, 15 |
| number of cases | Moderate | 0, 0 | 0, 0 |
| | Severe | 0, 0 | 0, 0 |
| Causal relationship with the | Definitely related | 0, 0 | 0, 0 |
| subject n, number of cases | Probably related | 0, 0 | 0, 0 |
| | Possibly related | 4, 4 | 0, 0 |
| | Probably not related | 0, 0 | 2, 2 |
| | Definitely not related | 12, 18 | 11, 13 |
| | Unknown | 0, 0 | 0, 0 |
| Serious adverse events n (%) | Yes | 1 (2.50) | 0 (0.00) |
| | No | 39 (97.50) | 40 (100.00) |
| | p-value [1] | | 1.0000(F) |
| Dropout due to adverse | Yes | 0 (0.00) | 0 (0.00) |
| events n (%) | No | 40 (100.00) | 40 (100.00) |
| | p-value [1] | | — |

Four adverse events that could not be ruled out to be related to the food for clinical trial occur in the test group, all of which were digestive disorders. These adverse events include 1 case of ‘loose stool’ and 3 cases of ‘dyspepsia’, and all of the subjects continued and completed the trial. One adverse event of ‘dyspepsia’ was cured, and one adverse event of ‘loose stool’ and two adverse events of ‘dyspepsia’ could not be followed up since the subject's last visit, failed to determine the disappearance of the adverse event (Table 4).

TABLE 4

| | | Test group | Control group |
|---|---|---|---|
| SOC | PT | N = 40 | N = 40 |
| (System Organ | (Preferred | n(%), number of | n(%), number of |
| Class) | Term) | cases | cases |
| Gastrointestinal | | 4 (10.00), 4 | 0 (0.00), 0 |
| disorders | Diarrhoea | 1 (2.50), 1 | 0 (0.00), 0 |
| | Dyspepsia | 3 (7.50), 3 | 0 (0.00), 0 |
| Total | | 4 (10.00), 4 | 0 (0.00), 0 |

Therefore, as a result of safety analysis on the adverse event and statistical processing in 80 subjects for the clinical trial, there was no association between the test group and adverse events under all conditions.

As described above, since a specific part of the content of the present disclosure is described in detail, for those of ordinary skill in the art, it is clear that the specific description is only a preferred embodiment, and the scope of the present disclosure is not limited thereby. Thus, the substantial scope of the present disclosure will be defined by the appended claims and their equivalents.

Name of depositor: Korea Research Institute of Bioscience and Biotechnology (KRIBB), Deposit date: Jul. 14, 2021, Accession number: KCTC14637BP, the address of the KRIBB is 125 Gwahak-ro, Yuseong-gu, Daejeon 334141, Republic of Korea. The deposit was made under Budapest Treaty, and that all restrictions imposed by the depository will be irrevocably removed upon the granting of the patent. The deposit is hereby incorporated by reference in its entirety.

What is claimed is:

1. A method of preventing or treating a muscle disease, comprising:
administering a pharmaceutical composition for comprising a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient to a subject.

2. The method of claim 1, wherein, in terms of intestinal microbiota composition changes, the pharmaceutical composition increases Veillonella spp. which is a beneficial bacterium and decreases Shigella spp. which is a harmful bacterium.

3. The method of claim 2, wherein the Veillonella spp. is any one or more selected from the group consisting of Veillonella sp. S12025-13, Veillonella dispar, Veillonella nakazawae, and Veillonella atypica, and the Shigella spp. is any one or more selected from the group consisting of Shigella sonnei, Shigella boydii, Shigella dysenteriae, and Shigella flexneri.

4. The method of claim 1, wherein the muscle disease is muscular atrophy, sarcopenia, atony, muscular dystrophy, myasthenia gravis, or amyotrophic lateral sclerosis.

5. The method of claim 1, wherein the pharmaceutical composition strengthens or improves muscle strength of hamstring which is responsible for flexion among femoral muscles.

6. The method of claim 1, wherein the pharmaceutical composition increases muscle mass of people in their 50s with a significant decrease in muscle mass.

7. A method of preventing or ameliorating a muscle disease, comprising:
administering a health functional food composition comprising a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient to a subject.

8. The method of claim 7, wherein, in terms of intestinal microbiota composition changes, the health functional food composition increases Veillonella spp. which is a beneficial bacterium and decreases Shigella spp. which is a harmful bacterium.

9. The method of claim 7, wherein the muscle disease is muscular atrophy, sarcopenia, atony, muscular dystrophy, myasthenia gravis, or amyotrophic lateral sclerosis.

10. A method of improving muscle strength or increasing muscle mass, comprising:
administering a health functional food composition comprising a heat-treated fermented culture complex of a Lactiplantibacillus plantarum KM2 strain deposited under KCTC 14637BP, a concentrate thereof, a dried product thereof, a fermented metabolite thereof, or a mixture thereof as an active ingredient to a subject.

11. The method of claim 7, wherein the health functional food composition strengthens or improves muscle strength of hamstring which is responsible for flexion among femoral muscles.

12. The method of claim 7, wherein the health functional food composition increases muscle mass of people in their 50s with a significant decrease in muscle mass.

13. The method of claim 10, wherein the health functional food composition strengthens or improves muscle strength of hamstring which is responsible for flexion among femoral muscles.

14. The method of claim 10, wherein the health functional food composition increases muscle mass of people in their 50s with a significant decrease in muscle mass.
